(19) United States
(12) Patent Application Publication
(10) Pub. No.: US 2024/0401805 A1
(43) Pub. Date: Dec. 5, 2024

(54) Virtual Sensor for Combustion Furnace

(71) Applicant: Vitro Flat Glass LLC, Cheswick, PA (US)

(72) Inventors: Priyadarshi Mahapatra, Pittsburgh, PA (US); Russell W. Schrier, Oakmont, PA (US); Wei Xu, Cheswick, PA (US); Yu Jiao, Blawnox, PA (US)

(21) Appl. No.: 18/660,630

(22) Filed: May 10, 2024

Related U.S. Application Data
(60) Provisional application No. 63/469,695, filed on May 30, 2023.

(51) Int. Cl. F23N 5/00; F23N 3/00; G06F 30/20

(52) U.S. Cl. CPC F23N 5/006; F23N 3/002; G01N 33/0068; G06F 30/20; F23N 2900/05001; F23N 2900/05002; F23N 2900/05003

(57) ABSTRACT

A method for operating a furnace includes: providing a furnace comprising feed materials, an oxygen-containing stream, and a fuel-containing stream; providing a virtual sensor including a model generated using inputs including condition data collected by a physical gas sensor previously arranged in the furnace; placing the virtual sensor in communication with the furnace; operating the furnace by a combustion reaction, where during operation of the furnace: the virtual sensor receives the further condition data; in response to receiving the further condition data, the virtual sensor, using the model, determines the combustion product based on the further condition data; and based on the determined combustion product, the virtual sensor sends a signal to the furnace to automatically adjust a flow rate of at least one input.

### CROSS-REFERENCE TO RELATED APPLICATIONS

This application claims priority to U.S. Provisional Application No. 63/469,695 filed May 30, 2023, the disclosure of which is hereby incorporated by reference in its entirety.

### BACKGROUND

### Field

The present disclosure is directed to a method for operating a furnace and, in some non-limiting embodiments or aspects, a method for operating a furnace using a virtual sensor. The present disclosure is also directed to a furnace system and a virtual sensor.

### Technical Considerations

Combustion furnaces often employ physical sensors arranged within the furnace to monitor operating conditions of the furnace. However, given the harsh conditions existing within the furnace (e.g., extreme temperature conditions), certain sensors exposed to these conditions quickly degrade and/or fail. For example, sensors for measuring oxygen composition can quickly degrade or fail in a combustion furnace. These sensors can be expensive, such that the short lifespan of these sensors caused by harsh combustion furnace conditions increases the cost of the production process.

### SUMMARY OF THE DISCLOSURE

According to some non-limiting aspects of the disclosure, a method for operating a furnace using a virtual gas sensor includes: providing a furnace including a first input including feed materials configured to be heated in the furnace, a second input including an oxygen-containing stream, and a third input including a fuel-containing stream; providing a virtual sensor including a model that determines a combustion product in the furnace, the model generated using inputs including condition data, the condition data including operating conditions of the furnace collected by a physical gas sensor previously arranged in the furnace; placing the virtual sensor in communication with the furnace such that the virtual sensor is configured to receive further condition data associated with operating conditions of the furnace; operating the furnace by a combustion reaction created by combining the oxygen from the oxygen-containing stream and the fuel from the fuel-containing stream, the combustion reaction heating the feed materials in the furnace, where during operation of the furnace: the virtual sensor receives the further condition data; in response to receiving the further condition data, the virtual sensor, using the model, determines the combustion product based on the further condition data; and based on the determined combustion product, the virtual sensor sends a signal to the furnace to automatically adjust a flow rate of at least one of the first input, the second input, and the third input.

In some non-limiting aspects, the method may further include: arranging the physical gas sensor in the furnace; operating the furnace by the combustion reaction during a training period before providing the virtual sensor; during the training period, collecting, with the physical gas sensor, the condition data; and generating the model using the condition data collected during the training period. The combustion product may include at least one of oxygen, CO, CO₂, NOx, and uncombusted fuel composition. The feed materials may include glass batch materials. The physical sensor may be arranged in a crown and/or a regenerator of the furnace to collect the condition data, and a reading of the physical gas sensor may not be an input to determine the combustion product in the furnace at a time during the operation of the furnace. The condition data may include at least one of fuel flow, oxygen flow, air flow, furnace temperature, oxygen composition, carbon dioxide composition, and carbon monoxide composition. The further condition data may not include a composition of at least one of oxygen, CO, CO₂, NOx, and uncombusted fuel, and the virtual sensor may determine the composition of the at least one of oxygen, CO, CO₂, NOx, and uncombusted fuel based on the further condition data. The model may be generated based on the condition data and reaction chemistry. The signal sent to the furnace may automatically adjust an oxygen-containing stream flow rate to the furnace.

In some non-limiting aspects, the method may further include: during the operation of the furnace, activating the physical sensor in the furnace while the virtual sensor is receiving the further condition data; determining, with the physical sensor, a measured combustion product in the furnace; and comparing the measured combustion product to the combustion product determined by the virtual sensor to determine a deviation. The method may further include: in response to the deviation being above a threshold, updating the model using the measured combustion product. At a time during the operation of the furnace, the furnace may include at least one of a fuel flow sensor, oxygen flow sensor, air flow sensor, and a temperature sensor, and the furnace does not use a reading of the physical gas sensor as an input to determine the combustion product in the furnace. The method may further include: providing a plurality of virtual sensors including a first virtual sensor including a first model and a second virtual sensor including a second model, where the first model is generated using inputs including condition data associated with operating conditions of a first location in the furnace, and the second model is generated using inputs including condition data associated with operating conditions of a second location in the furnace.

According to some non-limiting aspects of the disclosure, a furnace system includes: a furnace including a first input including feed materials configured to be heated in the furnace, a second input including an oxygen-containing stream, and a third input including a fuel-containing stream, the furnace configured to be operated by a combustion reaction created by combining the oxygen from the oxygen-containing stream and the fuel from the fuel containing stream; and a virtual sensor including a model that determines a combustion product in the furnace, the model generated using inputs including condition data, the condition data including operating conditions of the furnace collected by a physical gas sensor previously arranged in the furnace, where the virtual sensor is in communication with the furnace such that the virtual sensor is configured to receive further condition data associated with operating conditions of the furnace, where the virtual sensor is configured to determine the combustion product of the furnace in response to receiving the further condition data, where the virtual sensor is configured to send a signal to the furnace to automatically adjust a flow rate of at least one of the first input, the second input, and the third input based on the determined combustion product.

In some non-limiting aspects, the system may further include at least one of a fuel flow sensor, oxygen flow sensor, air flow sensor, and a temperature sensor arranged in the furnace. At a time during operation of the furnace, the furnace system may not use a reading of the physical gas sensor as an input to determine the combustion product in the furnace. The combustion product in the furnace may include at least one of oxygen, CO, CO₂, NOx, and uncombusted fuel composition. The feed materials may include glass batch materials. The condition data may include at least one of fuel flow, oxygen flow, air flow, furnace temperature, oxygen composition, carbon dioxide composition, and carbon monoxide composition. The further condition data may not include a composition of at least one of oxygen, CO, CO₂, NOx, and uncombusted fuel, and the virtual sensor determines the composition of the at least one of oxygen, CO, CO₂, NOx, and uncombusted fuel based on the further condition data. The model may be generated based on the condition data and reaction chemistry. The signal sent to the furnace may be configured to automatically adjust an oxygen-containing stream flow rate to the furnace.

In some non-limiting aspects, the model may be configured to be updated by: activating the physical sensor in the furnace while the virtual sensor is receiving the further condition data; determining, with the physical sensor, a measured combustion product in the furnace; comparing the measured combustion product to the combustion product determined by the virtual sensor to determine a deviation; and in response to the deviation being above a threshold, updating the model using the measured combustion product. A plurality of virtual sensors may be provided including a first virtual sensor including a first model and a second virtual sensor including a second model, where the first model is generated using inputs including condition data associated with operating conditions of a first location in the furnace, and the second model is generated using inputs including condition data associated with operating conditions of a second location in the furnace.

According to some non-limiting aspects of the disclosure, a virtual sensor includes at least one processor storing a model that determines a combustion product in a furnace, the model generated using inputs including condition data, the condition data including operating conditions of the furnace collected by a physical gas sensor previously arranged in the furnace.

In some non-limiting aspects, the furnace may include a first input including feed materials configured to be heated in the furnace, a second input including an oxygen-containing stream, and a third input including a fuel-containing stream, the furnace configured to be operated by a combustion reaction created by combining the oxygen from the oxygen-containing stream and the fuel from the fuel containing stream. The at least one processor may be programmed or configured to: communicate with the furnace so as to receive further condition data associated with operating conditions of the furnace; determine the combustion product of the furnace in response to receiving the further condition data; and send a signal to the furnace to automatically adjust a flow rate of at least one of the first input, the second input, and the third input based on the determined combustion product. At a time during operation of the furnace, the furnace system may not use a reading of the physical gas sensor as an input to determine the combustion product in the furnace. The combustion product in the furnace may include at least one of oxygen, CO, CO₂, NOx, and uncombusted fuel composition. The feed materials may include glass batch materials. The condition data may include at least one of fuel flow, oxygen flow, air flow, furnace temperature, oxygen composition, carbon dioxide composition, and carbon monoxide composition. The further condition data may not include a composition of at least one of oxygen, CO, CO₂, NOx, and uncombusted fuel, and the at least one processor is configured to determine the composition of the at least one of oxygen, CO, CO₂, NOx, and uncombusted fuel based on the further condition data. The model may be generated based on the condition data and reaction chemistry. The signal sent to the furnace may be configured to automatically adjust an oxygen-containing stream flow rate to the furnace. The at least one processor may be further programmed or configured to: while the virtual sensor is receiving the further condition data, receive a measured combustion product in the furnace determined by the physical sensor in the furnace; compare the measured combustion product to the combustion product determined by the virtual sensor to determine a deviation; and in response to the deviation being above a threshold, update the model based on the measure gas content.

According to some non-limiting aspects of the disclosure, a plurality of virtual sensors described herein form a system, the plurality of virtual sensors including a first virtual sensor including a first model and a second virtual sensor including a second model, where the first model is generated using inputs including condition data associated with operating conditions of a first location in the furnace, and the second model is generated using inputs including condition data associated with operating conditions of a second location in the furnace.

### BRIEF DESCRIPTION OF THE DRAWINGS

The disclosure will be described with reference to the following drawing figures wherein like reference numbers identify like parts throughout.

FIG. 1 shows a process flow diagram of a method for operating a furnace according to existing systems;

FIG. 2 shows a process flow diagram of a method for operating a furnace using a virtual sensor, according to some aspects of the disclosure;

FIG. 3 shows a process flow diagram of a method for generating a virtual sensor, according to some aspects of the disclosure;

FIG. 4 shows a schematic diagram of a furnace system according to existing systems;

FIG. 5 shows a schematic diagram of a furnace system having a virtual sensor, according to some aspects of the disclosure;

FIG. 6 shows a schematic diagram of a furnace system having a virtual sensor, according to some aspects of the disclosure;

FIG. 7 shows a schematic diagram of a model training system, according to some aspects of the disclosure;

FIG. 8 shows a schematic diagram of a model implementation system, according to some aspects of the disclosure;

FIG. 9 shows a schematic diagram of a model recalibration system, according to some aspects of the disclosure;

FIG. 10 shows a schematic diagram of a modeling process, according to some aspects of the disclosure; and

FIGS. 11-12B show test data from virtual sensors compared to physical sensors, according to some aspects of the disclosure.

### DETAILED DESCRIPTION

As used herein, spatial or directional terms, such as “left”, “right”, “inner”, “outer”, “above”, “below”, and the like, relate to the disclosure as it is shown in the drawing figures. However, it is to be understood that the disclosure can assume various alternative orientations and, accordingly, such terms are not to be considered as limiting. Further, as used herein, all numbers expressing dimensions, physical characteristics, processing parameters, quantities of ingredients, reaction conditions, and the like, used in the specification and claims are to be understood as being modified in all instances by the term “about”. Accordingly, unless indicated to the contrary, the numerical values set forth in the following specification and claims may vary depending upon the desired properties sought to be obtained by the present disclosure. At the very least, and not as an attempt to limit the application of the doctrine of equivalents to the scope of the claims, each numerical value should at least be construed in light of the number of reported significant digits and by applying ordinary rounding techniques. Moreover, all ranges disclosed herein are to be understood to encompass the beginning and ending range values and any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more and ending with a maximum value of 10 or less, e.g., 1 to 3.3, 4.7 to 7.5, 5.5 to 10, and the like. “A” or “an” refers to one or more.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or send (e.g., transmit) information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and transmits the processed information to the second unit. In some non-limiting embodiments or aspects, a message may refer to a network packet (e.g., a data packet and/or the like) that includes data.

Additionally, all documents, such as, but not limited to, issued patents and patent applications, referred to herein are to be considered to be “incorporated by reference” in their entirety.

The disclosure relates to a combustion furnace used in an industrial process. The industrial process can be any industrial process that can use an air and/or oxy-combustion furnace. For example, the furnace can be an air and/or oxy-combustion furnace used to manufacture glass.

A non-limiting background description of a furnace suitable for use in the present disclosure is as follows.

The furnace may be a large capacity furnace. For example, the furnace may be a glass production furnace. The furnace may include a first end or an opening where raw materials can to be fed into the furnace. Upon entering the furnace, the raw materials may be heated so as to be melted to form a molten material. The molten material can flow through a second end or discharge end.

The furnace may have a combustion chamber and a melting tank. The melting tank may be in communication with a feeder. The melting tank may be where the raw materials are melted. The feeder may hold unmelted raw materials, and provides the unmelted raw materials to the melting tank. Above the melting tank in the furnace may be a combustion chamber. The combustion chamber may comprise one or more burners that provide an oxygen-containing stream and a fuel-containing stream (e.g., a carbon-based fuel) to combust thereby providing heat to melt the materials in the melting tank. The combustion chamber and melting tank may form a melter crown as shown and described herein.

In one non-limiting example, the raw material may be raw glass material (also referred to as “glass batch materials”). The raw glass material may be placed into the furnace at the first end or opening to the furnace by a charging device or feeder. Inside the furnace, the raw glass material may be melted to form molten glass. The molten glass may flow out of the discharge into a fining zone.

A burner may be positioned in openings in the sidewalls of the furnace. The furnace typically has at least two sidewalls—a first sidewall and a second sidewall—wherein the first sidewall is opposite the second sidewall. The sidewall may have openings configured to receive the burner. The burner may be configured to provide an oxygen-containing gas, fuel and/or a mixture of oxygen-containing gas and fuel to the furnace, wherein the fuel combusts to form a flame. The flame from the burner provides the energy to melt the raw material. The burners may extend through a wall of the furnace or through a ceiling of the furnace. The furnace may have burners on one sidewall, or on the first and second sidewalls. The furnace may have at least 4, at least 6, at least 8, at least 10, or at least 12 burners; and/or at most 30 burners, at most 24 burners, at most 20 burners, or at most 16 burners. The burners on the first sidewall can be staggered (as opposed to in line) with the burners on the second sidewall.

The furnace may be an air and/or oxy-combustion furnace. The burners may be placed on the side walls at a certain distance from the surface of the molten material to provide suitable distribution of energy to melt the raw glass materials. For example, the burners can be at least 0.25 m, or at least 0.40 m, and less than 1.0 m or 0.8 m from the surface of the molten material.

The oxygen-containing gas may comprise pure oxygen, air, or some other oxygen-nitrogen gas blend.

The fuel-containing stream may comprise any hydrocarbon commonly burned in industrial furnaces. Non-limiting examples of fuel-containing streams include natural gas, fuel oil, coke, coal, or diesel.

The combustion process may form an exhaust gas in the combustion chamber. The exhaust gas may contain NOx gases. The NOx gases may be formed by oxygen reacting with nitrogen gas. The reaction may occur due to the high temperatures present in the furnace.

While furnaces used in glass production have been described herein, it will be appreciated that furnaces used in other applications are also within the scope of the present disclosure, such as furnaces used in metallurgical applications, furnaces used to conduct chemical reactions, furnaces used in hydrocarbon refining, and the like.

The present disclosure is directed to a method for operating a furnace, such as a furnace comprising any of the forgoing components, using a virtual gas sensor. The method may comprise: providing a furnace comprising a first input comprising feed materials configured to be heated in the furnace, a second input comprising an oxygen-containing stream, and a third input comprising a fuel-containing stream; providing a virtual sensor comprising a model that determines a combustion product in the furnace, the model generated using inputs comprising condition data, the condition data comprising operating conditions of the furnace collected by a physical gas sensor previously arranged in the furnace; placing the virtual sensor in communication with the furnace such that the virtual sensor is configured to receive further condition data associated with operating conditions of the furnace; operating the furnace by a combustion reaction created by combining the oxygen from the oxygen-containing stream and the fuel from the fuel-containing stream, the combustion reaction heating the feed materials in the furnace, wherein during operation of the furnace: the virtual sensor receives the further condition data; in response to receiving the further condition data, the virtual sensor, using the model, determines the combustion product based on the further condition data; and based on the determined combustion product, the virtual sensor sends a signal to the furnace to automatically adjust a flow rate of at least one of the first input, the second input, and the third input.

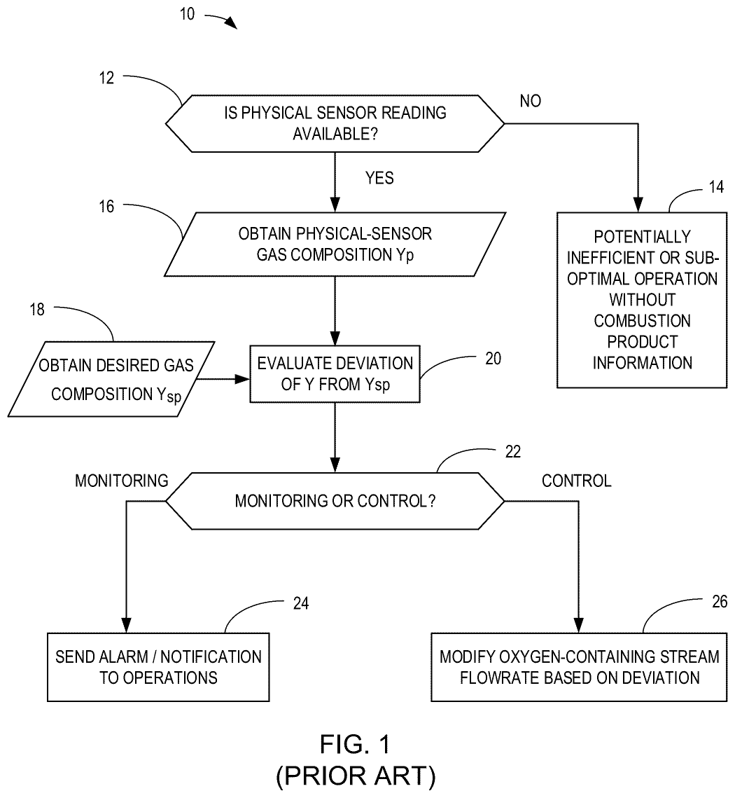

Referring to FIG. 1, a process 10 for operating a furnace according to existing systems is shown. The process 10 may comprise a step 12 of determining, with at least one processor (e.g., of a controller) whether a physical sensor is present and/or activated in the furnace to measure a combustion product of the furnace. A combustion product may refer to a composition of at least one compound generated and/or remaining in the furnace after the combustion reaction, such as at least one of oxygen, CO, CO₂, NOx, water and uncombusted fuel composition. The physical sensor may be configured to directly measure the combustion product in the furnace.

At a step 14, the processor may determine that no physical sensor is present and/or activated in the furnace to measure the combustion product of the furnace. In existing systems, the absence of such a physical sensor may indicate a potentially inefficient or sub-optimal operation in the absence of combustion product information. The present disclosure addresses such inefficiencies.

With continued reference to FIG. 1, at a step 16 the processor may determine that a physical sensor is present and/or activated in the furnace to measure the combustion product of the furnace. The physical sensor may directly measure the combustion product (Yₚ) in the furnace. At a step 18, the processor may determine a desired combustion product (Yₛₚ). The desired combustion product may be automatically determined by the processor and/or may be a user-specified set-point. At a step 20, the processor may compare the measured combustion product to the desired combustion product to determine a deviation of actual combustion product from desired combustion product.

With continued reference to FIG. 1, at a step 22, the processor may determine whether to initiate a monitoring and/or control protocol. At a step 24, the processor may initiate the monitoring protocol. The monitoring protocol may comprise the processor transmitting a notification and/or executing an alarm (e.g., visual and/or auditory alarm) to notify the user of the deviation of actual combustion product from desired combustion product. At a step 26, the processor may initiate the control protocol. The control protocol may comprise the processor automatically sending a control signal to adjust at least one component of the furnace. For example, the control signal may cause a flow valve to adjust (e.g., further open or close) to alter the flow rate of at least one of the oxygen-containing stream, fuel-containing stream, and/or feed materials stream to the furnace.

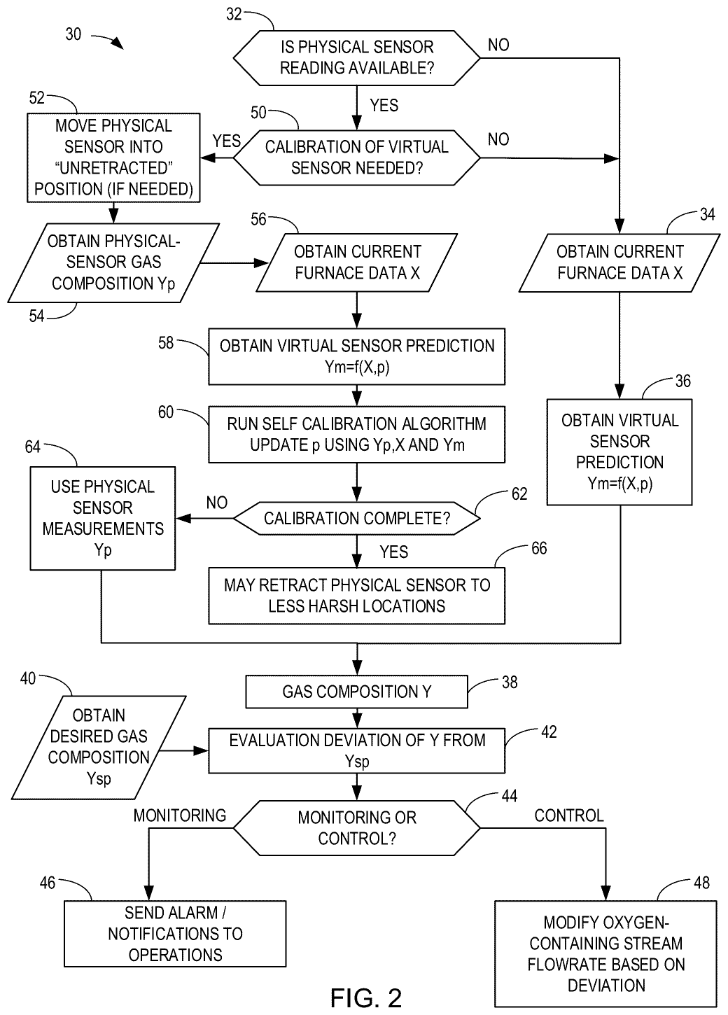

Referring to FIG. 2, a process 30 for operating a furnace using a virtual gas sensor is shown according to some embodiments or aspects. The process 30 may comprise a step 32 of determining, with the processor, whether a physical sensor is present and/or activated in the furnace to measure a combustion product of the furnace. Unlike the process 10 from FIG. 1, the absence and/or deactivation of a physical sensor in the furnace for directly measuring the combustion product does not preclude the combustion product from being determined as described herein.

With continued reference to FIG. 2, at a step 34, in response to determining that the physical sensor is not present and/or activated in the furnace, the processor may automatically obtain (e.g., receive and/or retrieve) further condition data associated with operation of the furnace (e.g., by the combustion reaction). The further condition data does not comprise a composition of at least one of oxygen, CO, CO₂, NOx, water and uncombusted fuel. In this way, the further condition data does not comprise the combustion product. The further condition data may comprise at least one of total gas flow, fuel flow, oxygen flow, air flow, and furnace temperature occurring in the furnace during operation thereof. The further condition data may be an indirect indicator of the combustion product, as determined by the model described herein. The further condition data may be measured by physical sensors configured to directly measure the further condition data. The physical sensors configured to measure the further condition data may be different from the physical sensor configured to measure the combustion product (which is not an input to the model in this embodiment).

At a step 36, the obtained further condition data may be input to the virtual sensor comprising a model as described herein. The virtual sensor may determine the combustion product (Yₘ) using the model based on the inputted further condition data. At a step 38, the determined combustion product determined by the virtual sensor may be considered the actual combustion product of the furnace.

At a step 40, the processor may determine the desired combustion product (Yₛₚ). At a step 42, the processor may compare the combustion product determined by the virtual sensor to the desired combustion product to determine a deviation of actual combustion product from desired combustion product.

At a step 44, the processor may determine whether to initiate a monitoring and/or control protocol. At a step 46, the processor may initiate the monitoring protocol. The monitoring protocol may comprise the processor transmitting a notification and/or executing an alarm (e.g., visual and/or auditory alarm) to notify the user of the deviation of actual combustion product from desired combustion product. At a step 48, the processor may initiate the control protocol. The control protocol may comprise the processor automatically sending a control signal to adjust at least one component of the furnace. For example, the control signal may cause a flow valve to adjust (e.g., further open or close) to alter the flow rate of at least one of the oxygen-containing stream, fuel-containing stream, and/or feed materials stream to the furnace.

With continued reference to FIG. 2, at a step 50, in response to determining that the physical sensor is present and/or activated in the furnace, the processor may automatically determine whether calibration of the virtual sensor should be conducted. If not, the process 30 may continue to step 34 as previously described. If so, the process 30 may continue to step 52.

At a step 52, the processor may cause the physical sensor to be positioned to directly measure the combustion product in the furnace. Positioning the physical sensor may include activating the physical sensor, physically moving the physical sensor from a first position to a second position (e.g., unretracting the physical sensor), removing a protective component from the physical sensor, or any combination thereof. Once positioned, at a step 54, the physical sensor may directly measure the combustion product (Yₚ) in the furnace.

At a step 56, the processor may automatically obtain (e.g., receive and/or retrieve) further condition data of the furnace. At a step 58, the obtained further condition data may be input to the virtual sensor comprising a model as described herein. The virtual sensor may determine the combustion product (Yₘ) using the model based on the inputted further condition data. At a step 60, the processor may recalibrate the virtual sensor (e.g., the model thereof) based on at least one of the further condition data, Yₚ, and Yₘ. During the recalibration, the Yₚ and further condition data directly measured by physical sensors are considered to be the actual combustion product and further condition data, and the Yₘ is considered to be the modeled combustion product, such that a deviation of the modeled combustion product from the actual combustion product is determined at step 60, and the model is recalibrated to more accurately predict the combustion product. This may include updating the algorithm (e.g., the parameters thereof and/or a weight of the parameters) on which the model generates the modeled combustion product Yₘ.

With continued reference to FIG. 2, at a step 62, the processor determines whether the calibration is completed. If yes, at a step 66, the physical sensor may be positioned to protect the physical sensor from the harsh conditions of the furnace. Positioning the physical sensor to protect the physical sensor may include deactivating the physical sensor, physically moving and/or removing the physical sensor from the second position to the first position (e.g., retracting the physical sensor or removing it altogether), emplacing a protective component on the physical sensor, or any combination thereof.

If step 62 determines that the calibration is not complete, at a step 64, the directly measured combustion product (Yₚ) in the furnace may be used as the actual combustion product, and the process 30 may continue to step 38 as previously described.

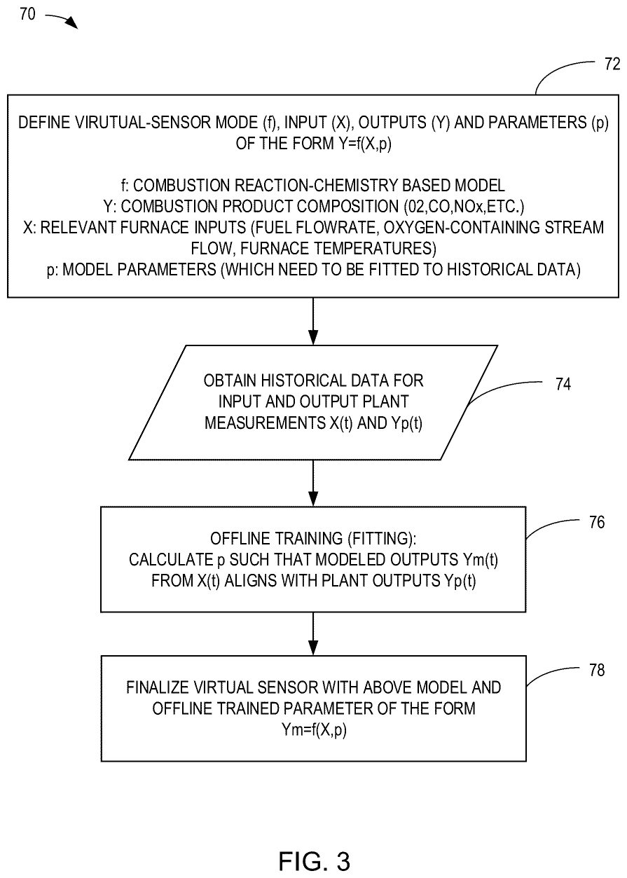

Referring to FIG. 3, a process 70 for generating a virtual gas sensor is shown according to some non-limiting embodiments or aspects. The process 70 may comprise a step 72 comprising determining features of the model of the virtual sensor. The features of the model may comprise reaction chemistry data associated with the combustion reaction (e.g., the chemical reactions and/or mass balances thereof), the inputs to the model (e.g., the condition data), the output of the model (e.g., the combustion product), and model parameters (e.g., parameters that need to be fit based on historical condition data). The model features, such as the inputs, parameters, and/or outputs, may be automatically determined by the processor (e.g., using a machine learning algorithm) and/or may be user specified or determined.

At a step 74, the processor may obtain (e.g., receive and/or retrieve) historical data. This historical data may comprise input and output data measured by physical sensors arranged in the furnace during a training period, as further described herein. The physical sensors during the training period may directly measure the combustion product and/or condition data of the furnace during the training period.

With continued reference to FIG. 3, the process 70 may comprise a step 76 of generating the model of the virtual sensor based on the obtained historical data. This may include fitting the parameters of the model based on the historical data to generate a model that accurately predicts combustion product based on the historical data. The analysis performed on the historical data to generate the model is not particularly limited and may including any type of analysis suitable to generate accurate results. Non-limiting examples of types of analysis that may be applied to the historical data include a non-linear regression analysis, such as a least square error non-linear regression analysis. At a step 78, the generated model may be finalized and implemented in the furnace after the training period.

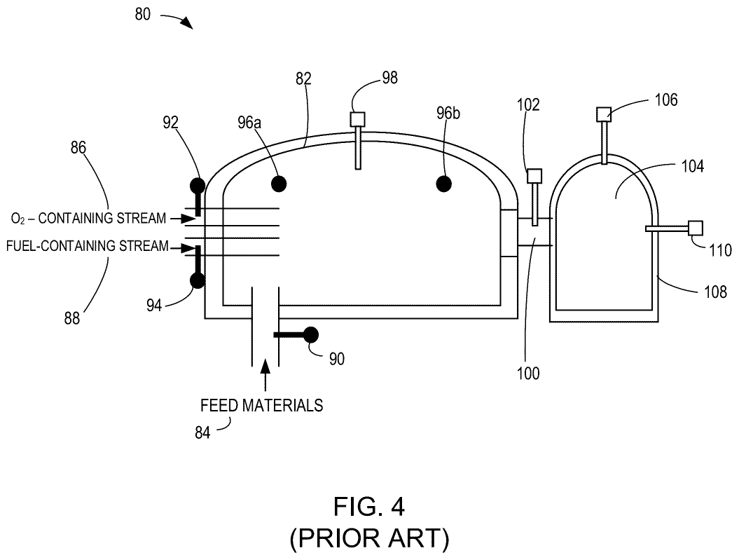

Referring to FIG. 4, a furnace system 80 is shown according to existing systems. While the present disclosure describes the specific design of a furnace system 80 shown in FIGS. 4-6, it will be appreciated that its design is non-limiting and other furnace designs are within the scope of the present disclosure.

The furnace system 80 of FIG. 4 may comprise a melter crown 82 in which the feed materials are fed and melted. The melter crown 82 may comprise a feed materials input 84 through which feed materials are fed to the melter crown 82. The feed materials may comprise any materials intended to be melted by the furnace. A non-limiting example of feed materials comprise glass batch materials used to make glass substrates. A non-limiting list of glass batch materials comprise at least one of SiO₂, Na₂O, CaO, MgO, Al₂O₃, K₂O, FeO, FezO₃, SO₃, Cr₂O₃, MnO, MnO₂, TiO₂, CeO₂, B₂O₃, NiO, Li₂O, CoO, V₂O₅, BaO, Cu, CuO, Co, Co₃O₄, Se, C, SnO₂, ZrO₂, Nd₂O₃, Er₂O₃, Sb₂O₃, As₂O₃, ZnO, NaNO₃, and the like. Other non-limiting examples of other raw materials that may be fed into the melter crown 82 include reactants of a chemical reaction, metal materials (e.g., for smelting), and hydrocarbons for refining.

The melter crown 82 may comprise an oxygen-containing stream input 86 through which the oxygen-containing stream is fed to the melter crown 82. The oxygen-containing stream may comprise pure oxygen, air, or some other oxygen-nitrogen gas blend.

The melter crown 82 may comprise a fuel-containing stream input 88 through which the fuel-containing stream is fed to the melter crown 82. The fuel may comprise any of the previously listed hydrocarbon fuels.

With continued reference to FIG. 4, each of the feed materials input 84, the oxygen-containing stream input 86, and the fuel-containing stream input 88 may comprise a respective flow meter 90, 92, 94 configured to directly measure the flow rate of the feed materials, oxygen-containing stream, and fuel-containing stream into the melter crown 82. Each of the feed materials input 84, the oxygen-containing stream input 86, and the fuel-containing stream input 88 may also comprise a valve (not shown) configured to control the amount of feed materials, oxygen-containing stream, and fuel-containing stream fed into the melter crown 82. The valves may be automatically controlled by a controller (not shown).

The melter crown 82 may also comprise temperature sensors 96a, 96b arranged therein to directly measure a temperature in a particular region in the furnace. The non-limiting example in FIG. 4 shows two temperature sensors 96a, 96b, including a first temperature sensor 96a measuring the temperature in the melter crown 82 in a first region and a second temperature sensor 96b measuring the temperature in the melter crown 82 in a second region. It will be appreciated that other embodiments may include only a single temperature sensor, while still other embodiments may include three or more temperature sensors.

The melter crown 82 may further comprise a physical sensor 98 configured to directly measure the combustion product in the melter crown 82. For example, the physical sensor 98 may be an oxygen sensor to directly measure oxygen composition in the melter crown 82. The combustion product may be any of those combustion products previously listed. While the physical sensor 98 in FIG. 4 is arranged in the crown of the melter crown 82, it will be appreciated that the physical sensor 98 may be positioned in one or more other regions of the melter crown 82 to directly measure the combustion product.

With continued reference to FIG. 4, the furnace system 80 may further comprise a port neck 100 comprising a port neck physical sensor 102 configured to directly measure combustion product therein. The port neck 100 may connect the melter crown 82 to one or more regenerator crowns 104.

The furnace system 80 may further comprise the regenerator crown 104 comprising a regenerator crown physical sensor 106 configured to directly measure combustion product therein. The regenerator crown 104 may comprise a target wall 108 comprising a target wall physical sensor 110 configured to directly measure combustion product at the target wall 108.

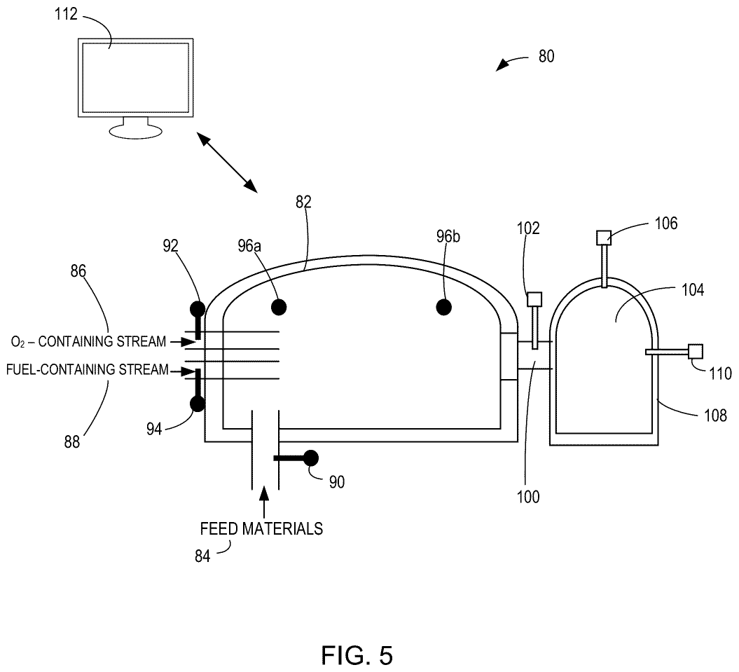

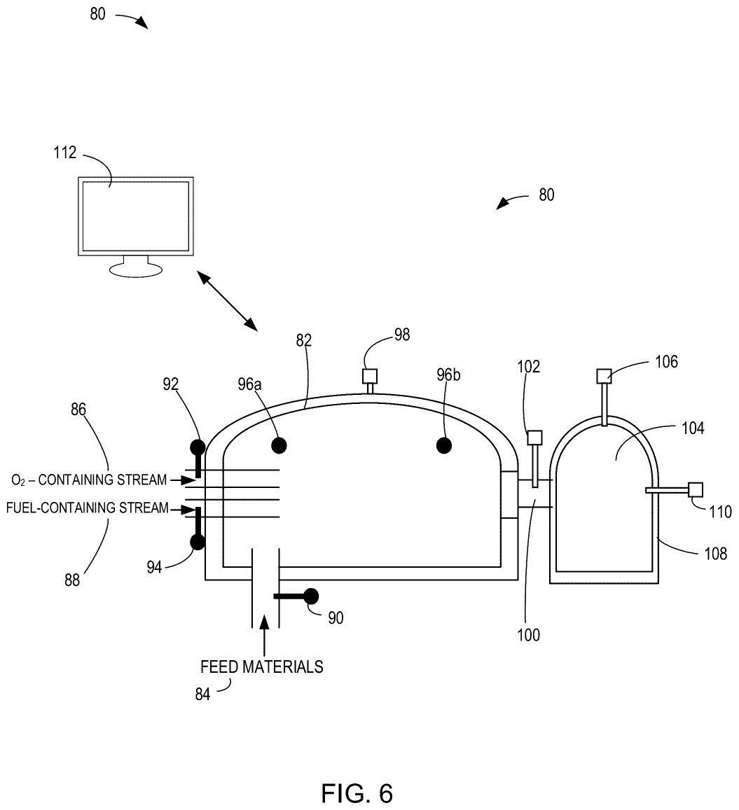

Referring to FIGS. 5 and 6, furnace systems 80 having a virtual gas sensor 112 (referred to interchangeably as “virtual sensor 112”) are shown according to some aspects of the disclosure. The furnace systems 80 of FIGS. 5 and 6 may include the same or similar components as the furnace system 80 of FIG. 4 except as described hereinafter. The furnace systems 80 of FIGS. 5 and 6 may further comprise the virtual sensor 112 in communication with a controller (not shown) of the furnace system 80. The virtual sensor 112 may replace and/or augment at least one physical sensor of the furnace system 80 configured to directly measure combustion product. While the non-limiting examples of FIG. 5 or 6 show the virtual sensor 112 replacing and/or augmenting the physical sensor 98 of the melter crown 82, it will be appreciated that the virtual sensor 112 may additionally or alternatively replace and/or augment the physical sensor 102, 106, 110 in at least one of the port neck 100, the regenerator crown 104, and the target wall 108.

Referring to FIG. 5, the furnace system 80 may comprise the virtual sensor 112 with the physical sensor 98 (see FIG. 4), removed from the melter crown 82. The physical sensor 98 may be fully removed from the melter crown 82 so as to protect the physical sensor 98 from the harsh conditions in the melter crown 82 and thus extend the life of the physical sensor 98. In some non-limiting embodiments, the physical sensor 98 may be re-inserted into the melter crown 82 periodically to evaluate the deviation of the virtual sensor 112 and/or to recalibrate the virtual sensor 112.

Referring to FIG. 6, the furnace system 80 may comprise the virtual sensor 112 with the physical sensor 98 retracted from the melter crown 82 (compared to FIG. 4). The physical sensor 98 being retracted from the melter crown 82 may protect the physical sensor 98 from the harsh conditions in the melter crown 82 and thus extend the life of the physical sensor 98. In some non-limiting embodiments, the physical sensor 98 may be unretracted into the melter crown 82 periodically to evaluate the deviation of the virtual sensor 112 and/or to recalibrate the virtual sensor 112.

With continued reference to FIGS. 5 and 6, while the physical sensor 98 is shown fully removed (FIG. 5) or retracted (FIG. 6) with the virtual sensor 112 configured to indirectly measure the combustion product based on the model, other arrangements of the physical sensor 98 are within the scope of this disclosure. For example, the physical sensor 98 may remain in the position shown in FIG. 4, but merely deactivated in favor of the virtual sensor 112. In some non-limiting embodiment, the physical sensor 98 and the virtual sensor 112 may be used to measure or model combustion product simultaneously, such as during recalibration of the model. In some non-liming embodiments, the physical sensor 98 may remain in the position shown in FIG. 4, but the physical sensor 98 may be covered by a separate component to protect the physical sensor from the harsh conditions in the melter crown 82.

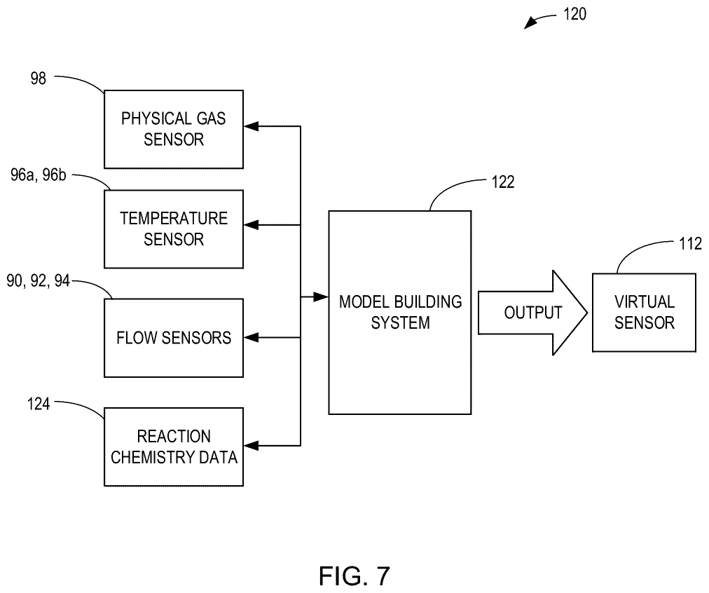

Referring to FIG. 7, a model training system 120 is shown, according to some aspects of the disclosure, which model training system 120 may be used to generate the virtual sensor 112 for use with the furnace systems 80 from FIGS. 5 and 6. The model training system 120 may comprise a model building system 122 comprising at least one processor configured to generate the virtual sensor 112 comprising the model based on inputs to the model building system 122. The virtual sensor 112 generated by the model building system 122 may be configured to determine a combustion product in the furnace.

With continued reference to FIGS. 5-7, the inputs to the model building system 122 may comprise condition data comprising operating conditions of the furnace collected by the physical sensor 98 previously arranged in the furnace (e.g., the melter crown 82; as used herein “furnace” and “melter crown” may be referred to interchangeably). Operating conditions of the furnace 82 may be collected by the physical sensor 98 previously arranged in the furnace 82 during a training period, such as by measuring the combustion product during the training period. The physical sensor 98 may be arranged during the training period in a crown of the furnace 82, the regenerator crown 104, or any other suitable position of the furnace system 80.

During the training period (before provision of the virtual sensor 112), the physical sensor 98 may be arranged in the furnace 82, and the furnace 82 may be operated by a combustion reaction of the oxygen-contain stream combusting the fuel-containing stream to heat and/or melt feed materials. During the training period, the physical sensor 98 may collect condition data of the furnace 82, such as by measuring a combustion product in the furnace 82 during the training period. During the training period, other physical sensors in the furnace system, such as the meters 90, 92, 94, the temperature sensors 96a, 96b, or other physical sensors (e.g., 102, 106, 110) may measure condition data of the furnace system 80. Non-limiting examples of condition data that may be measured during the training period may comprise at least one of fuel flow, oxygen flow, air flow, furnace temperature, oxygen composition, carbon dioxide composition, and carbon monoxide composition.

The condition data measured during this training period may be considered historical data, and this historical data may be input to the model building system 122. Reaction chemistry data 124 associated with the combustion reaction may also be input to the model building system 122. The reaction chemistry data 124 may comprise data associated with conducting the combustion reaction, such as the chemical equations conducted during the combustion reaction, including amounts (e.g., mols) of each reactant used and/or product formed during the combustion reaction. The reaction chemistry data 124 may comprise a mass balance of the combustion reaction.

Based on the foregoing inputs, the model building system 122 may generate a model comprising an algorithm that accurately predicts a combustion product based on historical input data. This may include determining the inputs that accurately predict the combustion product and/or fitting parameters that accurately predict the combustion product. The model building system 122 may comprise a machine learning algorithm and/or use any other suitable fitting programs. The output of the model building system 122 may comprise the virtual sensors 112 comprising the model.

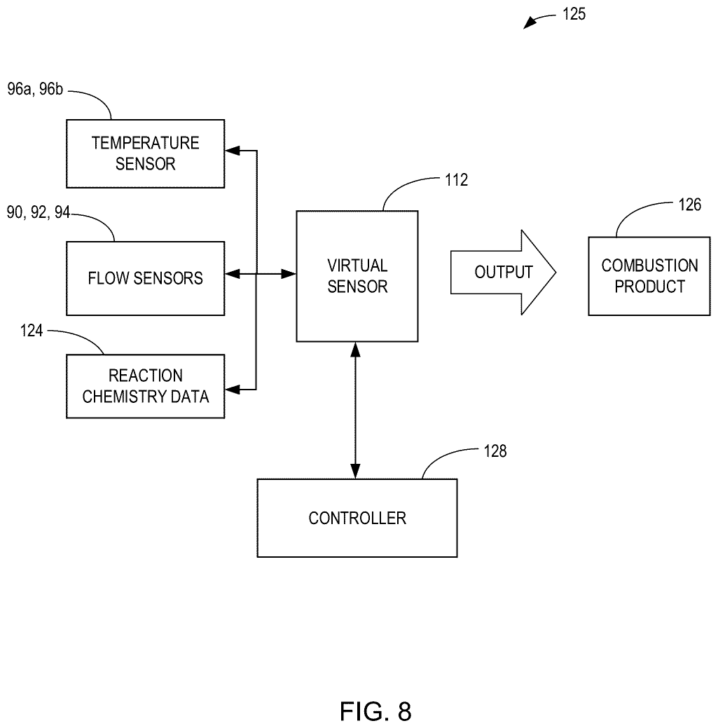

Referring to FIG. 8, a model implementation system 125 is shown according to some aspects of the disclosure, which model implementation system 125 may be used as a component of the furnace systems 80 from FIGS. 5 and 6. Integration of the implementation system 125 with the furnace system 80 may be done by providing the furnace system 80 described herein and integrating the virtual sensor 112 as a component of the furnace system 80 by providing the generated virtual sensor 112 to the furnace system 80.

Referring to FIGS. 5, 6, and 8, the virtual sensor 112 may be placed in communication with the furnace system 80, such as a controller 128 thereof. The controller 128 may be in communication with, for example, the physical sensor 98 (when activated), the flow meters 90, 92, 94, the temperature sensors 96a, 96b, other physical sensors (e.g., 102, 106, 110), valves (not shown) in the feed materials input 84, the oxygen-containing stream input 86, and the fuel-containing stream input 88, and the like.

With continued reference to FIGS. 5, 6, and 8, the furnace system 80 may be operated by a combustion reaction (e.g., in the melter crown 82) created by combining the oxygen from the oxygen-containing stream and the fuel from the fuel-containing stream heating the feed materials in the melter crown 82. Heating the feed materials may comprise melting the feed materials. During this operation of the furnace (after the training period and with the virtual sensor 112 integrated and operational), the virtual sensor 112 may receive the further condition data (as previously defined). For example, the virtual sensor 112 may receive measured data during operation of the furnace from the flow meters 90, 92, 94, the temperature sensors 96a, 96b, other physical sensors (e.g., 102, 106, 110), valves (not shown) in the feed materials input 84, the oxygen-containing stream input 86, and the fuel-containing stream input 88, and the like. The virtual sensor 112 may also receive reaction chemistry data 124 associated with the combustion reaction of the furnace system 80. Therefore, at a time during the operation of the furnace, the furnace system 80 may comprise at least one of a fuel flow sensor 94, oxygen or air flow sensor 92, and a temperature sensor 96a, 96b, and the furnace system 82 does not use a reading of the physical sensor 98 as an input to determine the combustion product 126 in the furnace system 80.

In response to receiving this further condition data, the virtual sensor 112, using the model, may automatically determine the combustion product 126 based on the input further condition data. In some non-limiting embodiments or aspects, the virtual sensor 112 determines the combustion product 126 without any input from the physical sensor 98, such that a reading of the physical sensor 98 is not an input to determine the combustion product 126 in the furnace system 80 at a time during the operation of the furnace 82. The combustion product 126 may comprise the virtual sensor 112 determining the composition of at least one of O₂, CO, CO₂, NOx, water, and uncombusted fuel based on the further condition data.

Based on the determined combustion product 126, the virtual sensor 112 may send a signal to the controller 128 of the furnace system 80 to automatically adjust at least one setting of the furnace system. For example, the controller 128 may automatically adjust a flow rate of at least one of the feed materials input 84, the oxygen-containing stream input 86, and the fuel-containing stream input 88, such as by causing a valve to further open or further close. For example, the controller 128 may automatically adjust a flow rate of the oxygen-containing stream input 86, to allow a higher or lower flow rate of oxygen to reach the furnace system 80.

The controller 128 may adjust at least one of the feed materials input 84, the oxygen-containing stream input 86, and the fuel-containing stream input 88 based on at least one predetermined threshold level of combustion product. For example, in response to the virtual sensor 112 determining that the combustion product is below a threshold level, the controller 128 may adjust at least one of the feed materials input 84, the oxygen-containing stream input 86, and the fuel-containing stream input 88 by taking a first action, and in response to the virtual sensor determining that the combustion product is above the threshold level, the controller 128 may adjust at least one of the feed materials input 84, the oxygen-containing stream input 86, and the fuel-containing stream input 88 by taking a second, different action. These adjustment may be automatically executed by the controller 128. Additionally or alternatively, the controller 128 may adjust a burner of the furnace based on at least one predetermined threshold level of combustion product.

For example, in response to the oxygen level in the combustion product being above a predetermined threshold, the controller 128 may decrease the oxygen-containing stream input 86 and/or increase the fuel-containing stream input 88 and/or adjust a burner of the furnace. For example, in response to the oxygen level in the combustion product being below a predetermined threshold, the controller 128 may increase the oxygen-containing stream input 86 and/or decrease the fuel-containing stream input 88 and/or adjust a burner of the furnace. For example, in response to the CO level in the combustion product being above a predetermined threshold, the controller 128 may increase the oxygen-containing stream input 86 and/or decrease the fuel-containing stream input 88 and/or adjust a burner of the furnace. For example, based on the NOx level in the combustion product, the controller 128 may adjust the oxygen-containing stream input 86 and/or the fuel-containing stream input 88 and/or adjust a burner of the furnace. For example, based on a ratio of CO:CO₂ in the combustion product, the controller 128 may adjust the oxygen-containing stream input 86 and/or the fuel-containing stream input 88 and/or adjust a burner of the furnace.

The furnace system 80 may operate continuously with the virtual sensor 112 continuously determining combustion product 126, and the virtual sensor 112 may communicate signals to the controller 128 to adjust at least one feature of the furnace as appropriate during this continuous operation of the furnace system 80.

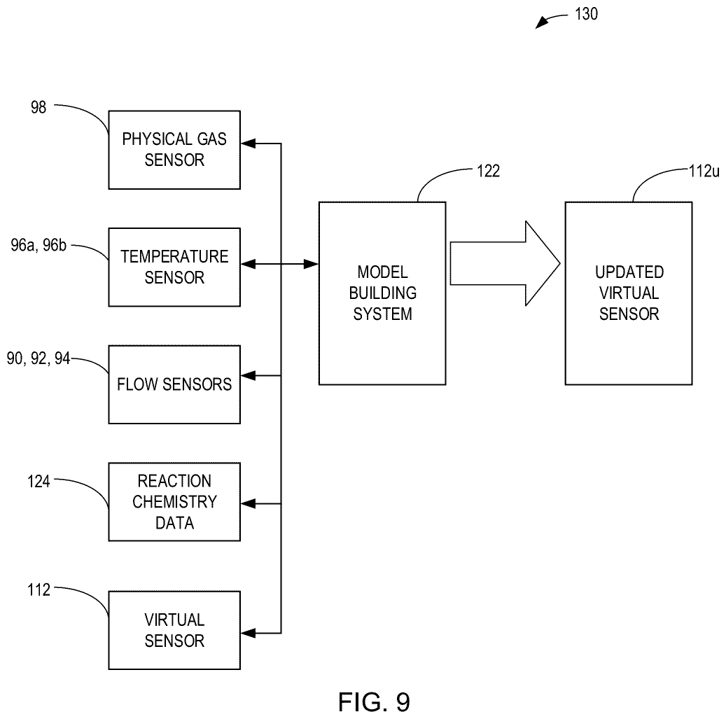

Referring to FIG. 9, a model recalibration system 130 is shown according to some aspects of the disclosure, which model recalibration system 130 may be used as a component of the furnace systems 80 from FIGS. 5 and 6. The model recalibration system 130 may be used to recalibrate (e.g., update) the previously-generated virtual sensor 112.

Referring to FIGS. 5, 6, and 9, the physical sensor 98 may be activated and/or reintroduced (e.g. inserted, unretracted, uncovered, and the like) into the furnace system 80 to recalibrate the virtual sensor 112. The physical sensor 98 and the virtual sensor 112 may be in communication with the model building system 122. The flow meters 90, 92, 94, the temperature sensors 96a, 96b, other physical sensors (e.g., 102, 106, 110), valves (not shown) in the feed materials input 84, the oxygen-containing stream input 86, and the fuel-containing stream input 88, and the like may be in communication with the model building system 122. The reaction chemistry data 124 may be in communication with the model building system 122.

During operation of the furnace to recalibrate the virtual sensor 112, the physical sensor 98 may be activated while the virtual sensor 112 is receiving further condition data and determining the combustion product. The physical sensor 98 may determine a measured combustion product by directly measuring the combustion product. The measured combustion product from the physical sensor 98 may be considered the actual combustion product while the combustion product determined by the virtual sensor 112 may be considered the modeled combustion product. The measured combustion product may be compared to the modeled combustion product to determine a deviation of the virtual sensor 112. It will be understood that the measured combustion product and the modeled combustion product correspond to the combustion product measured and modeled at the same time during operation of the furnace, such that the relevant combustion products are compared.

In response to determining the deviation, the model building system 122 may determine whether the deviation is above a threshold. In response to the deviation being above a threshold, the model building system 122 may update the model of the virtual sensor 112 based on the measured combustion product (measured by the physical sensor 98). The model may also be updated based on other inputs to the model building system 122, such as from at least one of the flow meters 90, 92, 94, the temperature sensors 96a, 96b, other physical sensors (e.g., 102, 106, 110), valves (not shown) in the feed materials input 84, the oxygen-containing stream input 86, and the fuel-containing stream input 88, the reaction chemistry data 124, and the like. The model building system 122 may generate the updated virtual sensor 112u comprising the updated model. The updated model may have parameters different (e.g., updated) from the previous model. The updated virtual sensor 112u may be integrated into the furnace system 80 and used to determine combustion product, such as when the physical sensor 98 is again deactivated and/or retracted and/or removed from the furnace system 80.

The virtual sensor 112 may be periodically recalibrated using the model recalibration system 130 as described herein to keep the virtual sensor 112 accurate. The virtual sensor 112 may be automatically recalibrated and/or recalibrated in response to a user executing a function requesting recalibration of the virtual sensor 112.

While the foregoing embodiments describe the furnace system 80 comprising a single virtual sensor, it will be appreciated that the furnace system 80 may comprise a plurality of virtual sensors, such as comprising a first virtual sensor comprising a first model and a second virtual sensor comprising a second, different model, although even further virtual sensors may be included. The virtual sensors may each be generated as previously described.

In some non-limiting embodiments or aspects, the first model may be generated using inputs comprising condition data associated with operating conditions of a first location in the furnace system, and the second model may be generated using inputs comprising condition data associated with operating conditions of a second location in the furnace system. For example, the first location in the furnace system may comprise the smelter crown while the second location may comprise at least one of the port neck, the regenerator crown, the target wall, and/or the like. For example the first location in the furnace system may comprise a first location in the smelter crown while the second location may comprise a second location of the smelter crown (e.g., two different locations within the same furnace component). The use of a plurality of virtual sensors may account for the variable conditions existing in different regions of the furnace and enable more accurate modeling of the combustion product by considering the conditions of the furnace system most influential in generating accurate models for combustion products in the different regions of the furnace. The different conditions in the furnace may be the result of one region being more proximate to or more remote from the location of the burners of the furnace firing the furnace, such as having a higher temperature on the right side of the furnace compared to the left side of the furnace when the furnace is firing from the right side. When generating the different models, different condition data in the furnace system may be used to generate the different models (e.g., as inputs to generating the models).

In some non-limiting embodiments, a single physical sensor arranged in the furnace system may be replaced by and/or augmented with a single virtual sensor. However, in some non-limiting embodiments, a single physical sensor arranged in the furnace system may be replaced by and/or augmented with a plurality of virtual sensors, such as when two different models enhance the accuracy of determining the combustion product previously measured by the single physical sensor.

The present disclosure is also directed to a virtual sensor comprising at least one processor storing a model that determines a combustion product in a furnace. The model may be generated as previously described, using inputs comprising the condition data comprising operating conditions of the furnace collected by a physical gas sensor previously arranged in the furnace.

The present disclosure is also directed to a plurality of virtual sensors comprising a first virtual sensor comprising a first model and a second virtual sensor comprising a second model, wherein the first model is generated using inputs comprising condition data associated with operating conditions of a first location in the furnace, and the second model is generated using inputs comprising condition data associated with operating conditions of a second location in the furnace.

The following numbered clauses are illustrative of various aspects of the disclosure:

Clause 1: A method for operating a furnace using a virtual gas sensor, comprising: providing a furnace comprising a first input comprising feed materials configured to be heated in the furnace, a second input comprising an oxygen-containing stream, and a third input comprising a fuel-containing stream; providing a virtual sensor comprising a model that determines a combustion product in the furnace, the model generated using inputs comprising condition data, the condition data comprising operating conditions of the furnace collected by a physical gas sensor previously arranged in the furnace; placing the virtual sensor in communication with the furnace such that the virtual sensor is configured to receive further condition data associated with operating conditions of the furnace; operating the furnace by a combustion reaction created by combining the oxygen from the oxygen-containing stream and the fuel from the fuel-containing stream, the combustion reaction heating the feed materials in the furnace, wherein during operation of the furnace: the virtual sensor receives the further condition data; in response to receiving the further condition data, the virtual sensor, using the model, determines the combustion product based on the further condition data; and based on the determined combustion product, the virtual sensor sends a signal to the furnace to automatically adjust a flow rate of at least one of the first input, the second input, and the third input.

Clause 2: The method of clause 1, further comprising: arranging the physical gas sensor in the furnace; operating the furnace by the combustion reaction during a training period before providing the virtual sensor; during the training period, collecting, with the physical gas sensor, the condition data; and generating the model using the condition data collected during the training period.

Clause 3: The method of clause 1 or 2, wherein the combustion product comprises at least one of oxygen, CO, CO₂, NOx, and uncombusted fuel composition.

Clause 4: The method of any of clauses 1-3, wherein the feed materials comprise glass batch materials.

Clause 5: The method of any of clauses 1-4, wherein the physical sensor is arranged in a crown and/or a regenerator of the furnace to collect the condition data, and a reading of the physical gas sensor is not an input to determine the combustion product in the furnace at a time during the operation of the furnace.

Clause 6: The method of any of clauses 1-5, wherein the condition data comprises at least one of fuel flow, oxygen flow, air flow, furnace temperature, oxygen composition, carbon dioxide composition, and carbon monoxide composition.

Clause 7: The method of clause 6, wherein the further condition data does not comprise a composition of at least one of oxygen, CO, CO₂, NOx, and uncombusted fuel, and the virtual sensor determines the composition of the at least one of oxygen, CO, CO₂, NOx, and uncombusted fuel based on the further condition data.

Clause 8: The method of any of clauses 1-7, wherein the model is generated based on the condition data and reaction chemistry.

Clause 9: The method of any of clauses 1-8, wherein the signal sent to the furnace automatically adjusts an oxygen-containing stream flow rate to the furnace.

Clause 10: The method of any of clauses 1-9, further comprising: during the operation of the furnace, activating the physical sensor in the furnace while the virtual sensor is receiving the further condition data; determining, with the physical sensor, a measured combustion product in the furnace; and comparing the measured combustion product to the combustion product determined by the virtual sensor to determine a deviation.

Clause 11: The method of clause 10, further comprising: in response to the deviation being above a threshold, updating the model using the measured combustion product.

Clause 12: The method of any of clauses 1-11, wherein at a time during the operation of the furnace, the furnace comprises at least one of a fuel flow sensor, oxygen flow sensor, air flow sensor, and a temperature sensor, and the furnace does not use a reading of the physical gas sensor as an input to determine the combustion product in the furnace.

Clause 13: The method of any of clauses 1-12, comprising: providing a plurality of virtual sensors comprising a first virtual sensor comprising a first model and a second virtual sensor comprising a second model, wherein the first model is generated using inputs comprising condition data associated with operating conditions of a first location in the furnace, and the second model is generated using inputs comprising condition data associated with operating conditions of a second location in the furnace.

Clause 14: A furnace system, comprising: a furnace comprising a first input comprising feed materials configured to be heated in the furnace, a second input comprising an oxygen-containing stream, and a third input comprising a fuel-containing stream, the furnace configured to be operated by a combustion reaction created by combining the oxygen from the oxygen-containing stream and the fuel from the fuel containing stream; and a virtual sensor comprising a model that determines a combustion product in the furnace, the model generated using inputs comprising condition data, the condition data comprising operating conditions of the furnace collected by a physical gas sensor previously arranged in the furnace, wherein the virtual sensor is in communication with the furnace such that the virtual sensor is configured to receive further condition data associated with operating conditions of the furnace, wherein the virtual sensor is configured to determine the combustion product of the furnace in response to receiving the further condition data, wherein the virtual sensor is configured to send a signal to the furnace to automatically adjust a flow rate of at least one of the first input, the second input, and the third input based on the determined combustion product.

Clause 15: The system of clause 14, further comprising at least one of a fuel flow sensor, oxygen flow sensor, air flow sensor, and a temperature sensor arranged in the furnace.

Clause 16: The system of clause 14 or 15, wherein at a time during operation of the furnace, the furnace system does not use a reading of the physical gas sensor as an input to determine the combustion product in the furnace.

Clause 17: The system of any of clauses 14-16, wherein the combustion product in the furnace comprises at least one of oxygen, CO, CO₂, NOx, and uncombusted fuel composition.

Clause 18: The system of any of clauses 14-17, wherein the feed materials comprise glass batch materials.

Clause 19: The system of any of clauses 14-18, wherein the condition data comprises at least one of fuel flow, oxygen flow, air flow, furnace temperature, oxygen composition, carbon dioxide composition, and carbon monoxide composition.

Clause 20: The system of clause 19, wherein the further condition data does not comprise a composition of at least one of oxygen, CO, CO₂, NOx, and uncombusted fuel, and the virtual sensor determines the composition of the at least one of oxygen, CO, CO₂, NOx, and uncombusted fuel based on the further condition data.

Clause 21: The system of any of clauses 14-20, wherein the model is generated based on the condition data and reaction chemistry.

Clause 22: The system of any of clauses 14-21, wherein the signal sent to the furnace is configured to automatically adjust an oxygen-containing stream flow rate to the furnace.

Clause 23: The system of any of clauses 14-22, wherein the model is configured to be updated by: activating the physical sensor in the furnace while the virtual sensor is receiving the further condition data; determining, with the physical sensor, a measured combustion product in the furnace; comparing the measured combustion product to the combustion product determined by the virtual sensor to determine a deviation; and in response to the deviation being above a threshold, updating the model using the measured combustion product.

Clause 24: The system of any of clauses 14-23, comprising: providing a plurality of virtual sensors comprising a first virtual sensor comprising a first model and a second virtual sensor comprising a second model, wherein the first model is generated using inputs comprising condition data associated with operating conditions of a first location in the furnace, and the second model is generated using inputs comprising condition data associated with operating conditions of a second location in the furnace.

Clause 25: A virtual sensor comprising at least one processor storing a model that determines a combustion product in a furnace, the model generated using inputs comprising condition data, the condition data comprising operating conditions of the furnace collected by a physical gas sensor previously arranged in the furnace.

Clause 26: The virtual sensor of clause 25, wherein the furnace comprises a first input comprising feed materials configured to be heated in the furnace, a second input comprising an oxygen-containing stream, and a third input comprising a fuel-containing stream, the furnace configured to be operated by a combustion reaction created by combining the oxygen from the oxygen-containing stream and the fuel from the fuel containing stream.

Clause 27: The virtual sensor of clause 26, wherein the at least one processor is programmed or configured to: communicate with the furnace so as to receive further condition data associated with operating conditions of the furnace; determine the combustion product of the furnace in response to receiving the further condition data; and send a signal to the furnace to automatically adjust a flow rate of at least one of the first input, the second input, and the third input based on the determined combustion product.

Clause 28: The virtual sensor of any of clauses 25-27, wherein at a time during operation of the furnace, the furnace system does not use a reading of the physical gas sensor as an input to determine the combustion product in the furnace.

Clause 29: The virtual sensor of any of clauses 25-28, wherein the combustion product in the furnace comprises at least one of oxygen, CO, CO₂, NOx, and uncombusted fuel composition.

Clause 30: The virtual sensor of any of clauses 25-29, wherein the feed materials comprise glass batch materials.

Clause 31: The virtual sensor of any of clauses 25-30, wherein the condition data comprises at least one of fuel flow, oxygen flow, air flow, furnace temperature, oxygen composition, carbon dioxide composition, and carbon monoxide composition.

Clause 32: The virtual sensor of any of clauses 27-31, wherein the further condition data does not comprise a composition of at least one of oxygen, CO, CO₂, NOx, and uncombusted fuel, and the at least one processor is configured to determine the composition of the at least one of oxygen, CO, CO₂, NOx, and uncombusted fuel based on the further condition data.

Clause 33: The virtual sensor of any of clauses 25-32, wherein the model is generated based on the condition data and reaction chemistry.

Clause 34: The virtual sensor of any of clauses 27-33, wherein the signal sent to the furnace is configured to automatically adjust an oxygen-containing stream flow rate to the furnace.

Clause 35: The virtual sensor of any of clauses 27-34, wherein the at least one processor is further programmed or configured to: while the virtual sensor is receiving the further condition data, receive a measured combustion product in the furnace determined by the physical sensor in the furnace; compare the measured combustion product to the combustion product determined by the virtual sensor to determine a deviation; and in response to the deviation being above a threshold, update the model based on the measure gas content.

Clause 36: A plurality of virtual sensors of any of clauses 25-35, the plurality of virtual sensors comprising a first virtual sensor comprising a first model and a second virtual sensor comprising a second model, wherein the first model is generated using inputs comprising condition data associated with operating conditions of a first location in the furnace, and the second model is generated using inputs comprising condition data associated with operating conditions of a second location in the furnace.

### EXAMPLES

### Example 1

### Generating a Model for a Virtual Sensor

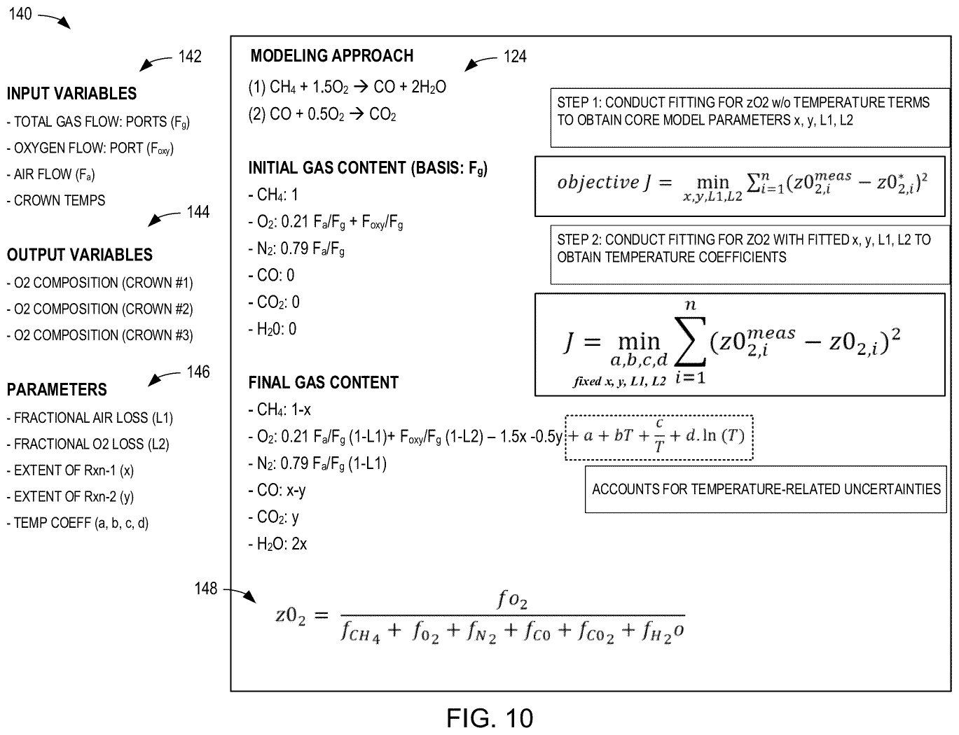

Referring to FIG. 10, a non-limiting example of a modeling process 140 is shown according to some aspects of the disclosure. The modeling process 140 may be used to generate a model for a virtual sensor. The modeling process 140 may include determining reaction chemistry data 124 of the combustion reaction, such as the reactions occurring in the combustion reaction and the mass balances thereof.

The modeling process 140 may include determining the input variables 142 to be input to the modeling building system to generate the model. The modeling process 140 may include determining the output variables 144 desired to be determined by the model (e.g., the combustion product). The modeling process 140 may include determining the parameters 146 which enable an accurate generation of the combustion product, which may include determining the most relevant parameters and/or the weight of those parameters. The modeling process 140 may output a model, which may comprise an algorithm 148 for determining a combustion product when integrated into the furnace system based on measured input parameters.

### Example 2

### Testing a Melter Crown Having a Single Virtual Sensor

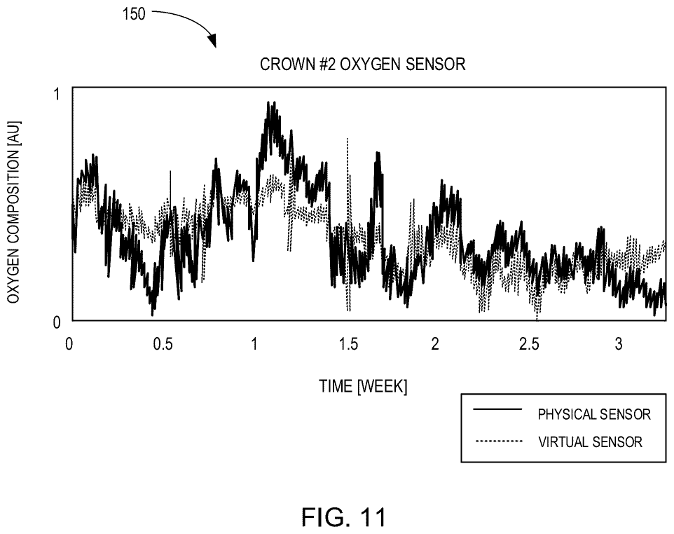

Referring to FIG. 11, a virtual sensor was generated according to the present disclosure for a melter crown (Crown #2), and the results from the generated virtual sensor tested against a physical oxygen (e.g., the combustion product) sensor arranged in the melter crown. A single virtual sensor was generated to represent the single physical oxygen sensor or averaging measurements from a single physical sensor over different operating conditions. Test data 150 showing the measured oxygen composition from the physical oxygen sensor was plotted over a span of several weeks compared to a modeled oxygen composition determined by the virtual sensor. As can be seen from the test data 150, the oxygen composition modeled by the virtual sensor closely matched the oxygen composition measured by the physical oxygen sensor over that same time, indicating that a highly accurate virtual sensor was generated to represent the readings of the physical oxygen sensor.

### Example 3

### Testing a Melter Crown Having Multiple Virtual Sensors

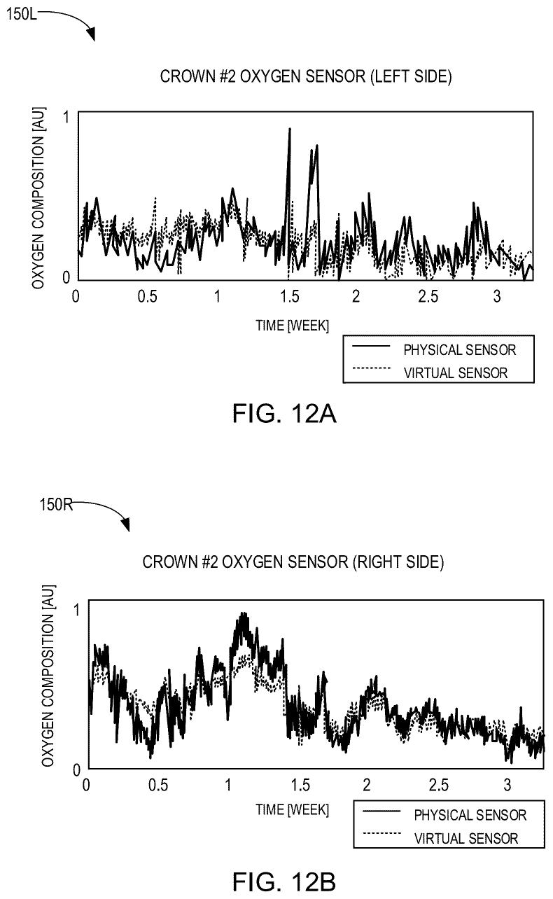

Referring to FIGS. 12A and 12B, a plurality of virtual sensors were generated according to the present disclosure for a melter crown (Crown #2), and the results from the generated virtual sensors tested against physical oxygen (e.g., the combustion product) sensors arranged in the melter crown. One virtual sensor was generated to represent a physical oxygen sensor arranged on a left side of the furnace or a physical oxygen sensor during firing from the left side, and a different virtual sensor was generated to represent a physical oxygen sensor arranged on a right side or a physical oxygen sensor during firing from the right side of the furnace.

Test data 150L shows the measured oxygen composition from the left physical oxygen sensor plotted over a span of several weeks compared to a modeled oxygen composition determined by the left virtual sensor. As can be seen from the test data 150L, the oxygen composition modeled by the left virtual sensor closely matched the oxygen composition measured by the left physical oxygen sensor over that same time, indicating that a highly accurate left virtual sensor was generated to represent the readings of the left physical oxygen sensor.

Test data 150R shows the measured oxygen composition from the right physical oxygen sensor plotted over a span of several weeks compared to a modeled oxygen composition determined by the right virtual sensor. As can be seen from the test data 150R, the oxygen composition modeled by the right virtual sensor closely matched the oxygen composition measured by the right physical oxygen sensor over that same time, indicating that a highly accurate right virtual sensor was generated to represent the readings of the right physical oxygen sensor.

It will be readily appreciated by those skilled in the art that modifications may be made to the invention without departing from the concepts disclosed in the foregoing description. Accordingly, the particular embodiments described in detail herein are illustrative only and are not limiting to the scope of the invention, which is to be given the full breadth of the appended claims and any and all equivalents thereof.

What is claimed is:

1. A method for operating a furnace using a virtual gas sensor, comprising:
providing a furnace comprising a first input comprising feed materials configured to be heated in the furnace, a second input comprising an oxygen-containing stream, and a third input comprising a fuel-containing stream;
providing a virtual sensor comprising a model that determines a combustion product in the furnace, the model generated using inputs comprising condition data, the condition data comprising operating conditions of the furnace collected by a physical gas sensor previously arranged in the furnace;
placing the virtual sensor in communication with the furnace such that the virtual sensor is configured to receive further condition data associated with operating conditions of the furnace;
operating the furnace by a combustion reaction created by combining the oxygen from the oxygen-containing stream and the fuel from the fuel-containing stream, the combustion reaction heating the feed materials in the furnace, wherein during operation of the furnace:
the virtual sensor receives the further condition data;
in response to receiving the further condition data, the virtual sensor, using the model, determines the combustion product based on the further condition data; and
based on the determined combustion product, the virtual sensor sends a signal to the furnace to automatically adjust a flow rate of at least one of the first input, the second input, and the third input.

2. The method of claim 1, further comprising:
arranging the physical gas sensor in the furnace;
operating the furnace by the combustion reaction during a training period before providing the virtual sensor;
during the training period, collecting, with the physical gas sensor, the condition data; and
generating the model using the condition data collected during the training period.

3. The method of claim 1, wherein the combustion product comprises at least one of oxygen, CO, CO₂, NOx, and uncombusted fuel composition.

4. The method of claim 1, wherein the feed materials comprise glass batch materials.

5. The method of claim 1, wherein the physical sensor is arranged in a crown and/or a regenerator of the furnace to collect the condition data, and a reading of the physical gas sensor is not an input to determine the combustion product in the furnace at a time during the operation of the furnace.

6. The method of claim 1, wherein the condition data comprises at least one of fuel flow, oxygen flow, air flow, furnace temperature, oxygen composition, carbon dioxide composition, and carbon monoxide composition.

7. The method of claim 6, wherein the further condition data does not comprise a composition of at least one of oxygen, CO, CO₂, NOx, and uncombusted fuel, and the virtual sensor determines the composition of the at least one of oxygen, CO, CO₂, NOx, and uncombusted fuel based on the further condition data.

8. The method of claim 1, wherein the model is generated based on the condition data and reaction chemistry.

9. The method of claim 1, wherein the signal sent to the furnace automatically adjusts an oxygen-containing stream flow rate to the furnace.

10. The method of claim 1, further comprising:
during the operation of the furnace, activating the physical sensor in the furnace while the virtual sensor is receiving the further condition data;
determining, with the physical sensor, a measured combustion product in the furnace; and
comparing the measured combustion product to the combustion product determined by the virtual sensor to determine a deviation.

11. The method of claim 10, further comprising:
in response to the deviation being above a threshold, updating the model using the measured combustion product.

12. The method of claim 1, wherein at a time during the operation of the furnace, the furnace comprises at least one of a fuel flow sensor, oxygen flow sensor, air flow sensor, and a temperature sensor, and the furnace does not use a reading of the physical gas sensor as an input to determine the combustion product in the furnace.

13. The method of claim 1, comprising:
providing a plurality of virtual sensors comprising a first virtual sensor comprising a first model and a second virtual sensor comprising a second model,
wherein the first model is generated using inputs comprising condition data associated with operating conditions of a first location in the furnace, and the second model is generated using inputs comprising condition data associated with operating conditions of a second location in the furnace.

14. A furnace system, comprising:
a furnace comprising a first input comprising feed materials configured to be heated in the furnace, a second input comprising an oxygen-containing stream, and a third input comprising a fuel-containing stream, the furnace configured to be operated by a combustion reaction created by combining the oxygen from the oxygen-containing stream and the fuel from the fuel containing stream; and
a virtual sensor comprising a model that determines a combustion product in the furnace, the model generated using inputs comprising condition data, the condition data comprising operating conditions of the furnace collected by a physical gas sensor previously arranged in the furnace,
wherein the virtual sensor is in communication with the furnace such that the virtual sensor is configured to receive further condition data associated with operating conditions of the furnace,
wherein the virtual sensor is configured to determine the combustion product of the furnace in response to receiving the further condition data,
wherein the virtual sensor is configured to send a signal to the furnace to automatically adjust a flow rate of at least one of the first input, the second input, and the third input based on the determined combustion product.

15. The system of claim 14, further comprising at least one of a fuel flow sensor, oxygen flow sensor, air flow sensor, and a temperature sensor arranged in the furnace.

16. The system of claim 14, wherein at a time during operation of the furnace, the furnace system does not use a reading of the physical gas sensor as an input to determine the combustion product in the furnace.

17. The system of claim 14, wherein the combustion product in the furnace comprises at least one of oxygen, CO, CO₂, NOx, and uncombusted fuel composition.

18. The system of claim 14, wherein the feed materials comprise glass batch materials.

19. A virtual sensor comprising at least one processor storing a model that determines a combustion product in a furnace, the model generated using inputs comprising condition data, the condition data comprising operating conditions of the furnace collected by a physical gas sensor previously arranged in the furnace.

20. A plurality of virtual sensors of claim 19, the plurality of virtual sensors comprising a first virtual sensor comprising a first model and a second virtual sensor comprising a second model,
wherein the first model is generated using inputs comprising condition data associated with operating conditions of a first location in the furnace, and the second model is generated using inputs comprising condition data associated with operating conditions of a second location in the furnace.
